(19) United States
(12) Patent Application Publication
(10) Pub. No.: US 2005/0284491 A1
(43) Pub. Date: Dec. 29, 2005

(54) Operating room control system

(75) Inventors: Koichi Tashiro, Sagamihara-shi (JP); Takeaki Nakamura, Tokyo (JP); Akinobu Uchikubo, Iruma-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, TOKYO (JP)

(21) Appl. No.: 11/157,448

(22) Filed: Jun. 21, 2005

(30) Foreign Application Priority Data
Jun. 21, 2004 (JP) 2004-182132

(51) Int. Cl. G05B 11/01; G06F 13/28; G06F 17/00; A61B 19/00

(52) U.S. Cl. 128/897; 700/90

(57) ABSTRACT

An operating room control system including: a first controller connected to a medical device placed in an operating room; a second controller connected to a nonmedical device placed in the operating room; and an operating instruction input device for accepting input of an operating instruction for the medical device and for transmitting the content of the entered operating instruction to the first controller. In this operating room control system, an operating instruction for the nonmedical device is also entered into the operating instruction input device. The first controller transmits a first control signal according to the operating instruction for the nonmedical device entered into the operating instruction input device to the second controller. The second controller converts the first control signal to a second control signal for controlling the nonmedical device and transmits it to the nonmedical device. Thereby, a surgeon can operate the nonmedical device by himself.

### CROSS-REFERENCE TO RELATED APPLICATION

This application is based upon and claims the benefit of priority from prior Japanese Patent Application No. 2004-182132, filed Jun. 21, 2004, the entire contents of which is incorporated herein by reference.

### BACKGROUND OF THE INVENTION

1. Field of the Invention

The present invention relates to an operating room control system, and more particularly to an operating room control system in which a surgeon or the like can operate nonmedical devices.

2. Description of the Related Art

Conventionally, there has been suggested a surgery system in which a system controller controls medical devices including an endoscope used in a surgery (Refer to FIG. 1 in Laid-Open Japanese Patent Publication No. 2004-157177, for example). The system controller is connected to, for example, an electric cautery, an insufflator, an endoscopic camera, and a light source as medical devices, which are controlled devices. The system controller is further connected to a display device and an operating panel. The operating panel, which comprises a display section and a touch sensor, is an integrated operating device operated by a nurse or the like in a nonsterilized area. Endoscopic images or the like are displayed on the display device.

On the other hand, there are nonmedical devices such as a room light, a room camera, an interphone, a liquid crystal display device, and other audio-visual equipment in the operating room. The audio-visual equipment is controlled by an audio-visual controller for conducting individual or integral controls.

The aforementioned surgery system, however, does not link with the nonmedical devices or audio-visual equipment. Therefore, if the surgeon wants to change the intensity of lighting in the room, for example, he has to instruct a nurse or other person to turn on the room light by voice so as to make the nurse operate an audio-visual controller. Therefore, if the surgeon wants to control a device in the operating room, he needs to make an instruction by voice every time to achieve a desired operation. Such oral instructions to a nurse or other person in the operating room adds to the complexity of the surgeon's work and the surgeon cannot control an audio-visual device by himself when he desires.

In view of the above problem, the present invention has been provided. Therefore, it is an object of the present invention to provide an operating room control system enabling a surgeon to operate nonmedical devices by himself with a surgery system linking with a nonmedical device system.

### BRIEF SUMMARY OF THE INVENTION

Accordingly, there is provided an operating room control system comprising: a first controller connected to one or more medical devices disposed in an operating room; a second controller connected to one or more nonmedical devices disposed in the operating room; and an operating instruction input device for accepting input of an operating instruction for the one or more medical devices and for transmitting the content of the entered operating instruction to the first controller. An operating instruction for the one or more nonmedical devices is also entered into the operating instruction input device. The first controller transmits a first control signal according to the operating instruction for the one or more nonmedical devices entered into the operating instruction input device to the second controller. The second controller converts the first control signal to a second control signal for controlling the one or more nonmedical devices and transmits the one or more to the nonmedical device.

Thereby, a surgeon can operate the one or more nonmedical devices by himself.

The nonmedical device can be, for example, a room light, a room camera, a ceiling camera, an image reference storage server, or an image recording device, each of which is in the operating room.

If an endoscope is used as the one or more medical devices, the operating instruction input device can be a manual operation button associated with the endoscope.

If the operating room control system according to the present invention further comprises a display member for displaying a content of an operating instruction of the operating instruction input device in the present invention, a surgeon can check the content of the instruction easily, thereby reducing operating errors.

The one or more nonmedical devices can include audio-visual devices, for example.

### BRIEF DESCRIPTION OF THE SEVERAL VIEWS OF THE DRAWINGS

These and other features, aspects, and advantages of the apparatus and methods of the present invention will become better understood with regard to the following description, appended claims, and accompanying drawings where:

FIG. 1 is a schematic diagram showing a a configuration of an endoscopic surgery system according to an embodiment of the present invention;

FIG. 2 is a block diagram showing a a configuration of an operating room control system according to the embodiment of FIG. 1;

FIG. 3 is a diagram for explaining control signals between the endoscopic surgery system and an audio-visual system of FIG. 2;

FIG. 4 is a flowchart showing an example of a processing flow of converting a control signal in an AV controller of FIG. 2; and

FIG. 5 is a diagram showing examples of commands displayed on a display screen of an operating panel.

### DETAILED DESCRIPTION OF THE PREFERRED EMBODIMENS

Preferred embodiments of the invention are described below with reference to the accompanying drawings.

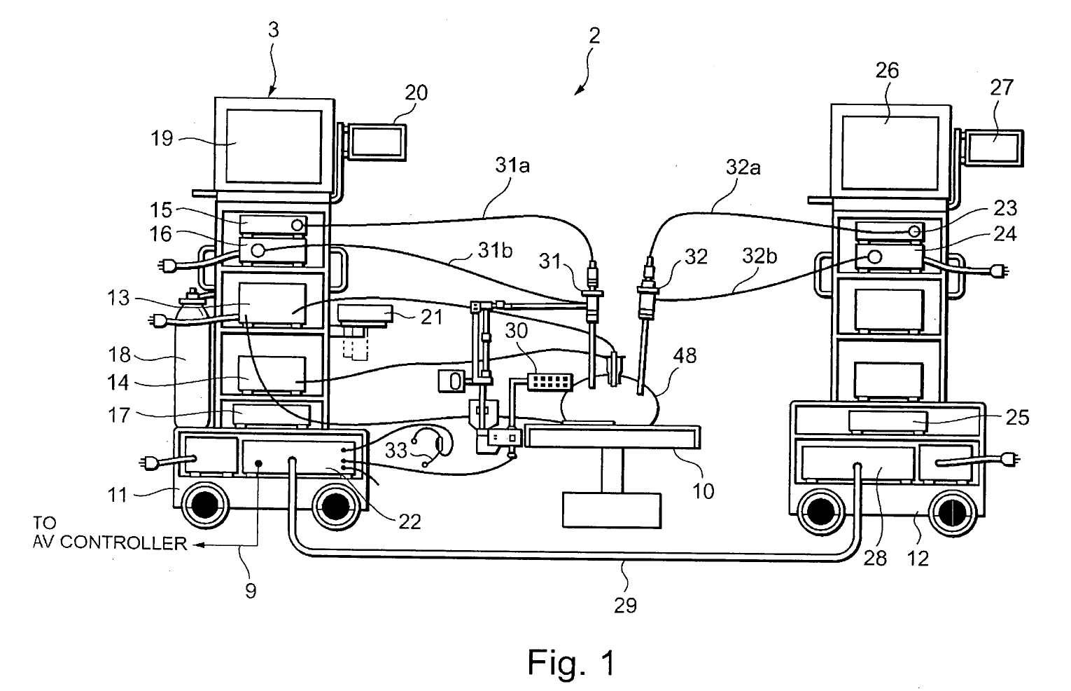

An operating room control system according to an embodiment comprises a plurality of medical devices and a system controller as a medical device controller. The description begins with an explanation of an endoscopic surgery system, which is one of the examples of a surgery system including a plurality of medical devices and a system controller for controlling these medical devices. Referring to FIG. 1, there is shown a schematic diagram illustrating a configuration of an endoscopic surgery system 3 placed in an operating room 2.

As shown in FIG. 1, a patient's bed 10 on which a patient 48 is laying and the endoscopic surgery system 3 are placed in the operating room 2. The endoscopic surgery system 3 has a first cart 11 and a second cart 12.

The first cart 11 is loaded with medical devices as controlled devices such as, for example, an electric cautery 13, an insufflator 14, an endoscopic camera 15, a light source 16, a video tape recorder (VTR) 17, and other devices and a gas cylinder 18 filled with carbon dioxide. The endoscopic camera 15 is connected to a first endoscope 31 via a camera cable 31a. The light source 16 is connected to the first endoscope 31 via a light guide cable 31b.

Moreover, the first cart 11 is loaded with a display device 19, a first integrated display 20, an operating panel 21, and the like. The display device 19 is, for example, a TV monitor for displaying endoscopic images or the like.

The integrated display panel 20 is a display member capable of selectively displaying various data during surgery. The operating panel 21 comprises a display section such as, for example, a liquid crystal display, and a touch sensor integrally arranged on the display section, for example. The operating panel 21 is an integrated operating device, which is operated by a nurse or the like in a nonsterilized area.

Furthermore, the first cart 11 is loaded with a system controller 22, which is a controller. The system controller 22 is connected to the electric cautery 13, the insufflator 14, the endoscopic camera 15, the light source 16, and the VTR stated above via communication lines (not shown). The system controller 22 can be connected to a headset-type microphone 33. Thereby, the system controller 22 can recognize voice input from the microphone 33 and can control devices according to a surgeon's voice.

On the other hand, the second cart 12 is loaded with an endoscopic camera 23, a light source 24, an image processing device 25, a display device 26, and a second integrated display panel 27, which are controlled devices.

The endoscopic camera 23 is connected to a second endoscope 32 via a camera cable 32a. The light source 24 is connected to the second endoscope 32 via a light guide cable 32b.

The display device 26 displays endoscopic images picked up by the endoscopic camera 23. The second integrated display panel 27 can selectively display various data during surgery.

The endoscopic camera 23, the light source 24, and the image processing device 25 are connected to a repeater unit 28 mounted on the second cart 12 via communication lines (not shown). The repeater unit 28 is connected to the system controller 22 mounted on the first cart 11 via a trunk cable 29.

The system controller 22 integrally controls the camera 23, the light source 24, and the image processing device 25 mounted on the second cart 12 and the electric cautery 13, the insufflator 14, the camera 15, the light source 16, and the VTR 17 mounted on the first cart 11. If the system controller 22 is communicating with these devices, the system controller 22 can display the setting conditions of the connected devices and the setting screens of control switches on the liquid crystal display of the operating panel 21. Moreover, the system controller 22 can enter an operation such as a change of a setting value through an operation on the touch sensor in a given area in response to a touch on the setting screen of a desired control switch.

A remote controller 30 is a second integrated operating device operated by a surgeon or the like in the sterilized area. It can control other devices, for which the communication is established, via the system controller 22.

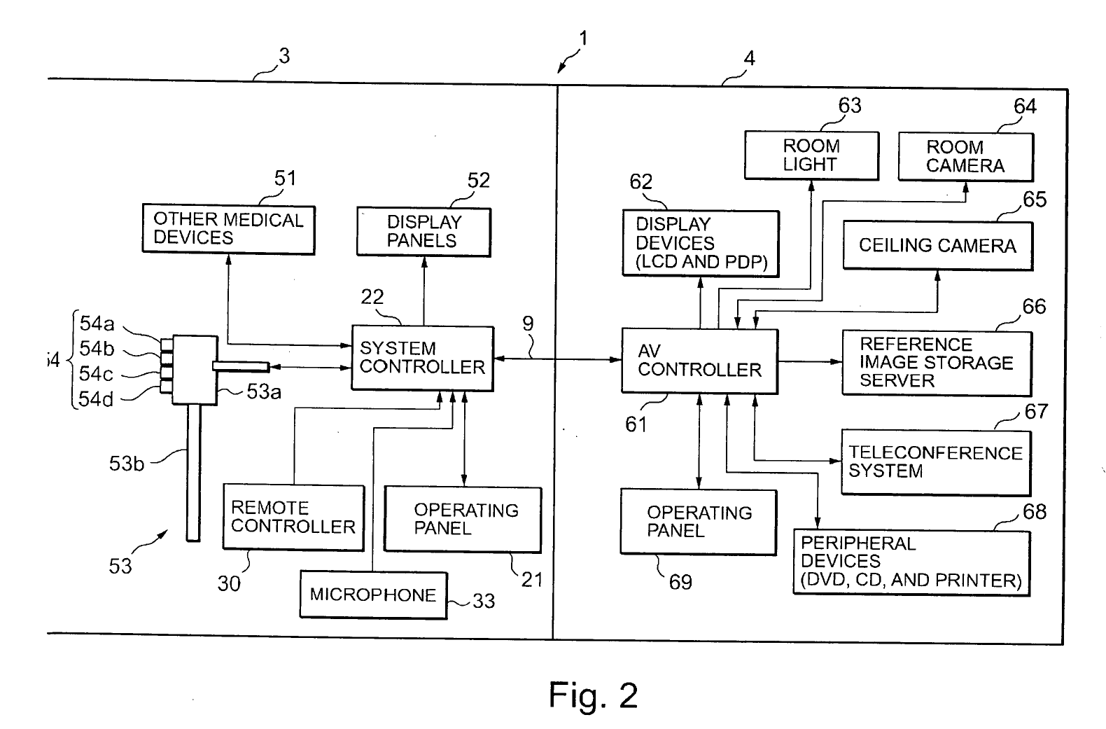

Referring to FIG. 2, there is shown a block diagram illustrating the entire configuration of the operating room control system 1 according to this embodiment. The operating room control system 1 comprises the endoscopic surgery system 3 and an audio-visual system (hereinafter, referred to as AV system) 4 as a nonmedical device system. The endoscopic surgery system 3 has a configuration as shown in FIG. 1. FIG. 2 shows a simplified configuration for presenting a brief description. In FIG. 2, other medical devices 51 include the electric cautery 13 or the like connected to the system controller 22 directly or indirectly via the repeater unit 28. Display panels 52 include the first integrated display panel 20 and the second integrated display panel 27 connected via the repeater unit 28.

In FIG. 2, an endoscope 53 is the first endoscope 31 or the second endoscope 32, which is connected to the system controller 22 directly or indirectly. The endoscope 53 comprises an operating section 53a and an insert section 53b. The operating section 53a is provided with a plurality of manual operation buttons 54, more specifically four manual operation buttons 54a to 54d. Moreover, as shown in FIG. 1, the system controller 22 is connected to a remote controller 30, a microphone 33, and an operating panel 21 as operating instruction input devices.

The audio-visual system 4 of the operating room control system 1 includes an audio-visual controller (hereinafter, referred to as AV controller) 61. The AV controller 61 is connected to the system controller 22 via a cable 9.

The AV controller 61 controls nonmedical devices including connected audio-visual devices (hereinafter, referred to as AV devices). As shown in FIG. 2, the nonmedical devices connected to the AV controller 61 can include a display device 62, a room light 63, a room camera 64, a ceiling camera 65, a reference image storage server 66, a conference system for a conference with remote conferees (hereinafter, referred to as a teleconference system) 67, and peripheral devices 68. Furthermore, the AV controller 61 is connected to an operating panel 69. Thus, the term “AV devices” means nonmedical devices including facility devices such as a lighting system placed in the operating room 2 as well as devices for recording or reproducing image data other than the endoscope 53.

The display device 62 can be a liquid crystal display (LCD) or a plasma display panel (PDP), which displays an image in a device previously specified or specified on the operating panel 69 by a nurse or the like, such as, for example, an image from the reference image storage server described later. The room light is a device for illuminating the inside of the operating room 2. The room camera 64 is for use in taking pictures of the inside of the operating room 2. The ceiling camera 65 is suspended from the ceiling of the operating room 2 and can be controlled to change a viewing position. The reference image storage server 66 is a server storing image data such as pre-surgery endoscopic images of a patient, ultrasound tomographic images, or image data of the PACS or the like, such as, for example, a DICOM server and is placed inside or outside the operating room 2. The teleconference system 67 permits a conversation with a nurse or the like in a medical office or a nurse station while displaying images with voice. The peripheral devices 68 can be, for example, a printer, a CD player, a DVD recorder, or other various devices. Endoscopic images of the endoscopic surgery system 3 are supplied to the AV system via a signal line other than the cable 9 and can be recorded by the DVD recorder. The operating panel 69 is similar to the operating panel 21 and is a device for controlling the AV devices connected to the AV controller 61.

The operating room control system 1 according to this embodiment is configured in such a way that the AV devices connected to the AV controller 61 are made controllable by operating the operating instruction input device such as the remote controller 30 connected to the system controller 22.

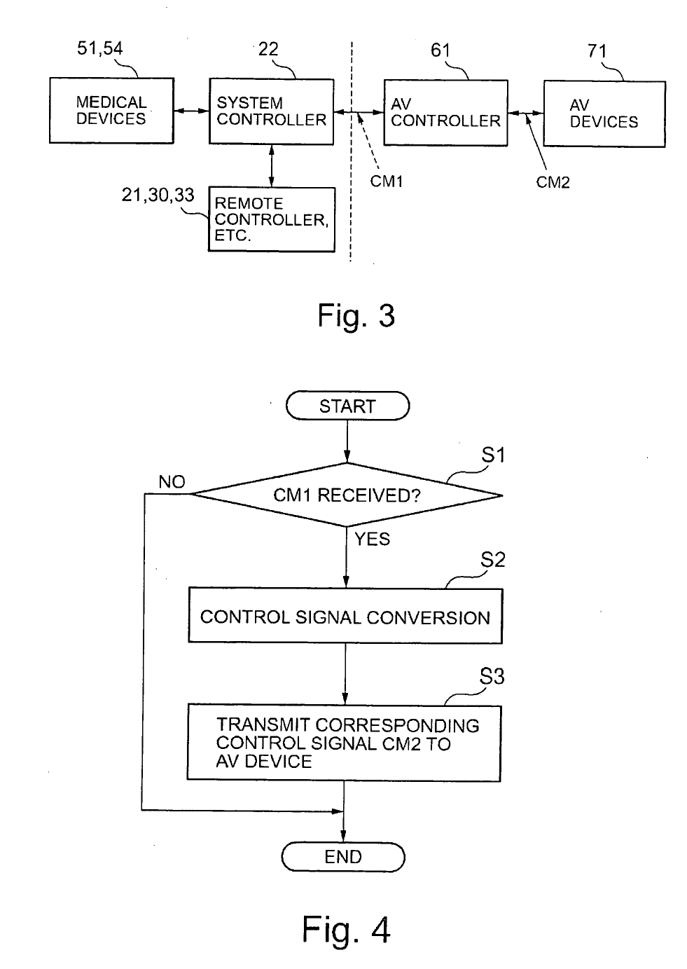

The following describes a method of controlling the AV devices from the remote controller 30 or the like by referring to FIG. 3. Referring to FIG. 3, there is shown a diagram for explaining control signals between the endoscopic surgery system 3 and the AV system 4.

As shown in FIG. 3, if an operating instruction is given to the medical devices 51 and 54 in the remote controller 30, the system controller 22 normally transmits the control signals to the medical devices 51 and 54 or performs processing for output signals from the medical devices 51 and 54. With the press of a given key on the remote controller 30, however, the system controller 22 transmits a control signal CM1 corresponding to the key to the AV controller 61 via the cable 9. The AV controller 61 transmits a control signal (hereinafter, referred to as corresponding control signal) CM 2, which corresponds to the control signal CM1 intended for AV devices, to the corresponding AV device. The system controller 22 may transmit the signal from the remote controller 30 as the control signal CM1 directly to the AV controller 61.

It is previously determined which control signals of the AV devices correspond to given buttons on the remote controller 30 or the like between the system controller 22 and the AV controller 61. In other words, a relation between the control signal CM1 and the control signal CM2, namely, information indicating what operation of which AV device the control signal CM1 corresponds to is previously determined between the system controller 22 and the AV controller 61. Therefore, with a given input, such as the press of a given key (button) on the remote controller 30, the AV controller 61 outputs a given manipulate signal of the AV device corresponding to the given input or button to the AV device.

More specifically, if the remote controller 30 is provided with a plurality of function keys, arbitrary commands can be set to the function keys. Therefore, for example, a command “record an endoscopic image using a DVD recorder” is previously allocated to a function key 1 (hereinafter, referred to as key F1). Thereafter, if a surgeon presses the key F1 during a surgery, the system controller 22 transmits the given control signal CM1 corresponding to the key F1 to the AV controller 61.

Upon receiving the control signal CM1 corresponding to the key F1 of the remote controller 30, the AV controller 61 transmits the control signal CM2 corresponding to the “record” command to the DVD recorder. The corresponding control signal CM2 carries the “record” command conforming to the DVD recorder command system and thus the endoscopic image is appropriately recorded in the DVD recorder included in the AV devices 71.

While the commands to the AV devices 71 are allocated to the function keys of the remote controller 30 in the above description, it is also possible to provide the remote controller 30 with dedicated keys for the AV devices 71, instead of the function keys, for issuing operating instructions.

The commands to the AV devices 71 that can be specified on the remote controller 30 can be: record, replay, and stop commands to the DVD recorder; replay and stop commands to the CD player; on and off commands to the room light; up and down commands for a light control of the room light; record, reproduce, and stop commands for images to the reference image storage server 66; on and off commands and volume up and down commands to the teleconference system 67; and on and off commands to internal phones. Furthermore, the record command to the DVD recorder may include a distinction between recording of an endoscopic image and recording of an in-room image.

While the above description has been made by giving an example of controlling the AV devices 71 using the function keys on the remote controller 30, the surgeon may use manual operation buttons 54 provided in the operating section 53a of the endoscope 53 if the surgeon is operating the endoscope 53. Depending on the endoscope type, it is possible to allocate given commands to the manual operation buttons 54 of the endoscope 53. For this type of endoscope, commands to the AV devices as stated above are previously allocated to the manual operation buttons 54a to 54d. Thereby, the surgeon can control the AV devices of the AV system 4 by operating the manual operation buttons 54 of the operating section 53a while operating the endoscope 53.

Referring to FIG. 4, there is shown a flowchart illustrating an example of a processing flow of converting a control signal in the AV controller 61. At step S1, the AV controller 61 determines whether it has received a control signal CM1 from the system controller 22 via the cable 9. Unless it has received the control signal CM1, S1 results in NO and the processing ends directly. If it has received the control signal CM1, the result is YES in S1 and thus the AV controller executes control signal converting processing at step S2. The control signal converting processing is performed using table data including a correspondence between the control signal CM1 and the corresponding control signal CM2 previously stored, for example. In the above example, the table data includes stored information that the control signal CM 1 of the key F1 corresponds to the corresponding control signal CM 2 of the command indicating recording with the DVD recorder. Therefore, the AV controller 61 can convert the control signal CM1 to the corresponding control signal CM2 on the basis of the table data.

Then, the AV controller 61 transmits the corresponding control signal CM2 to an AV device at step S3. Information on the AV device to which the corresponding control signal CM2 should be transmitted is previously stored so as to suit the above table data, for example. Therefore, the AV controller 61 transmits the corresponding control signal CM2 on the basis of the previously stored information for the control signal CM1 or the corresponding control signal CM2.

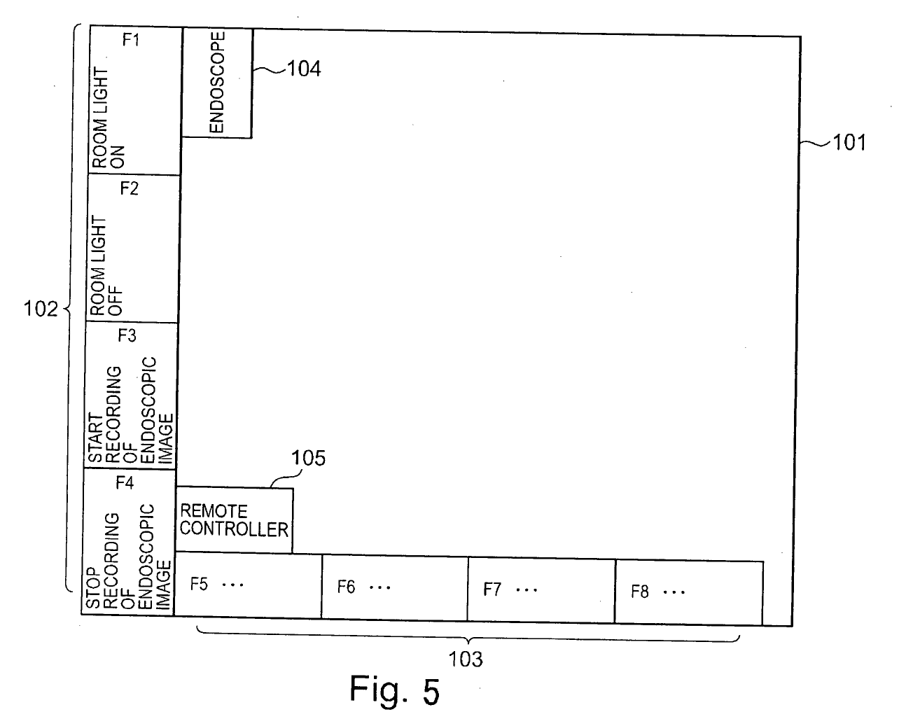

Moreover, the display device such as the display panel 52 or the operating panel 21 may display buttons indicating corresponding commands to show the surgeon the correspondence between them. Referring to FIG. 5, there is shown a diagram of an example of commands displayed on the display screen on the operating panel 21.

As shown in FIG. 5, the contents of the commands are displayed along with the function key numbers in given positions on the display screen 101 of the operating panel 21 (at the left and bottom ends of the screen in FIG. 5). At the left end of the screen, there is provided a display section 102 showing that the keys F1, F2, F3, and F4 correspond to “room light ON,“ “room light OFF,” “start recording of endoscopic image,” and “stop recording of endoscopic image,” respectively. Similarly, at the bottom end of the screen, there is provided a display section 103 showing what commands the keys F5 to F8 correspond to, respectively. Moreover, there is provided a display section 104 showing that the keys F1 to F4 are the manual operation buttons 54 of the endoscope 53 in the vicinity of the keys F1 to F4. Similarly, there is provided a display section 105 showing that the keys F5 to F8 are the function keys of the remote controller 30 in the vicinity of the keys F5 to F8.

Therefore, the operating panel 21 serving as a display member displays the contents of the operating instructions of the function keys. Thereby, the surgeon can easily ascertain which button corresponds to what command of which AV device by viewing the operating panel 21.

As stated hereinabove, a command is appropriately transmitted to the corresponding AV device on the basis of the control signal CM1 from the system controller 22.

While an operation command to an AV device has been entered using a button of the remote controller or the operating section of the endoscope in the above description, a surgeon may issue an operating instruction to the AV device by voice using the microphone 33. The system controller 22 may convert the voice input from the microphone to text by voice recognition, determine the content of the operating instruction on the basis of the converted content, generate a control signal CM1 on the basis of the determination result, and transmit the control signal CM1 to the AV controller 61.

Still further, the operating panel 21 may be used in such a way that a nurse can issue an operating instruction to the AV device. For example, the operating panel 21 displays buttons for controlling the AV devices of the AV system 4 on the screen. The operating panel 21 can be a touch panel. Therefore, with a touch on a displayed button, the system controller 22 can determine what instruction the operator has issued. For example, with a touch on the button for turning on the room light 63 on the operating panel 21, the system controller 22 transmits the ON signal of the room controller 63 to the AV controller 61.

If an operating instruction to the AV device is issued to the operating panel 21, the system controller 22 transmits a control signal CM1 according to the operating instruction to the AV controller 61 via the cable 9 as a communication path. Upon receiving the control signal CM1, the AV controller 61 transmits the corresponding control command CM2 to the AV device corresponding to the control signal CM1 to the AV device.

As described hereinabove, according to this embodiment, the surgery system links with the nonmedical device system and therefore the surgeon can operate both the medical devices and the nonmedical devices without assistance from a nurse or other person in the operating room.

Moreover, the surgery system links with the nonmedical device system and thus a surgeon can issue operating instructions to the devices of the nonmedical system. Therefore, when using a device in the area showing a significant technical advance such as an image recording apparatus, the latest image recording apparatus can be connected to the nonmedical device system for use.

If the operating device of the surgery system can control all nonmedical devices of the nonmedical device system, the operating panel of the nonmedical device system can be eliminated.

While there has been shown and described what are considered to be preferred embodiments of the invention, it will, of course, be understood that various modifications and changes in form or detail could readily be made without departing from the spirit of the invention. It is therefore intended that the invention not be limited to the exact forms described and illustrated, but constructed to cover all modifications that may fall within the scope of the appended claims.

1. An operating room control system comprising:
a first controller connected to one or more medical devices disposed in an operating room;
a second controller connected to one or more nonmedical devices disposed in the operating room; and
an operating instruction input device for accepting input of an operating instruction for the one or more medical devices and the one or more nonmedical devices and for transmitting the content of the entered operating instruction to the first controller, wherein:
the first controller has a function of transmitting a first control signal according to the operating instruction for the one or more nonmedical devices entered into the operating instruction input device to the second controller; and
the second controller has a function of converting the first control signal to a second control signal for controlling the one or more nonmedical devices and transmitting the second control signal to the one or more nonmedical devices.

2. The operating room control system according to claim 1, wherein:
the one or more nonmedical devices includes at least one of a room light, a room camera, a ceiling camera, an image reference storage server, and an image recording device.

3. The operating room control system according to claim 1, wherein the one or more medical devices comprises an endoscope.

4. The operating room control system according to claim 3, wherein the operating instruction input device is a manual operation button associated with the endoscope.

5. The operating room control system according to claim 1, further comprising a display member for displaying a content of an operating instruction of the operating instruction input device.

6. The operating room control system according to claim 1, wherein the one or more nonmedical devices comprises an audio-visual device.

7. A method of operating one or more nonmedical devices from an operating instruction input device for one or more medical devices, the method comprising:
accepting input of an operating instruction for the one or more nonmedical devices from the operating instruction input device by using a controller for the one or more medical devices;
transmitting the input of the operating instruction for the one or more nonmedical devices from the controller for the one or more medical devices to a controller for the one or more nonmedical devices;
converting the input of the operating instruction to an operation command for the one or more nonmedical devices to be operated by using the controller for the one or more nonmedical devices; and
transmitting the converted command from the controller for the one or more nonmedical devices to the one or more nonmedical devices to be operated.
